(19) United States
(12) Patent Application Publication
(10) Pub. No.: US 2011/0097359 A1
(43) Pub. Date: Apr. 28, 2011

(54) Production of diphtheria toxin

(76) Inventor: Tim Lee, Toronto (CA)

(21) Appl. No.: 10/582,576

(22) PCT Filed: Nov. 30, 2004
(86) PCT No.: PCT/CA2004/002024

§ 371 Date: Jul. 30, 2007

Related U.S. Application Data
(60) Provisional application No. 60/481,780, filed on Dec. 12, 2003.

(51) Int. Cl. A61K 39/05; C12N 1/20; C12P 21/00; C07K 14/34; A61P 37/04; A61P 31/04

(52) U.S. Cl. 424/238.1; 435/253.6; 435/71.3; 530/350

(57) ABSTRACT

A Corynebacterium diphtheriae culture medium for the production of diphtheria toxin and methods for producing the toxin are provided. The medium is substantially free of animal-derived products and comprises water, a carbohydrate source, a nitrogen source and a number of free amino acids in an initial concentration wherein the initial concentration of each free amino acid is not limiting for the production of the toxin.

### FIELD OF THE INVENTION

The present invention relates to a bacterial growth medium and a process for the production of diphtheria toxin.

### BACKGROUND OF THE INVENTION

Diphtheria is a life-threatening disease caused by infection with Corynebacterium diphtheriae, a gram-positive, aerobic, rod-shaped bacterium. The disease is caused by local invasion of nasopharyngeal tissues by toxin-producing strains of C. diphtheriae. The organisms grow in a tough, fibrinous membrane overlying a painful, hemorrhapic, and necrotic lesion, which may be located on the tonsils or within the nasopharynx region. During typical epidemics of the past, the spread of the disease was by droplet infection. Patients who recover from diphtheria may carry toxigenic bacteria in, their throats and nasopharynx for weeks or months, unless intensively treated with antibiotics.

Most of the clinical symptoms of diphtheria are due to the potent diphtheria toxin produced from corynebacterioprophage carrying the tox gene. After the prophage infects the C. diphtheriae strain and lysogenization has taken place, the strain becomes virulent. Toxin neutralizing antibodies (antitoxin) induced by active immunization with non-toxic forms (toxoids) of the diphtheria toxin can prevent diphtheria. The current immunization strategy is the utilization of diphtheria vaccines prepared by converting the diphtheria toxin into its non-toxic, but antigenic, toxoid form by formaldehyde treatment. The diphtheria toxoid is used in various combinations with other vaccine components for mass Immunization worldwide. The World Health Organization (WHO) recently estimated that about 100,000 cases worldwide and up to 8,000 deaths per year are due to decreased immunization of infants, waning immunity to diphtheria in adults and insufficient supply of vaccines.

The variant of the Parke Williams 8 (PW8) strain of Corynebacterium diphtheriae is often used to produce the exotoxin from which the toxoid is prepared by chemical modification. In general, a medium formulation with amino acids, trace vitamins, inorganic salts and a carbohydrate source such as maltose promotes excellent growth of the bacterium. Different media, such as the acid digest of casein and the enzymatic digest of beef muscle (trypsin or papain) are suitable media for toxin production. In conventional methods, the bacteria are cultivated in media containing proteinaceous material of animal origin. A commonly used medium in diphtheria production is the NZ-Amine Type A medium, which contains a casein digest. Under optimal conditions, the amount of toxin produced using NZ-Amine Type A media is 180 Lf/mL using the Limes of flocculation method. (References 1-3,—Throughout this application various references are referred to in parenthesis to more fully describe the sate of the art to which this invention pertains. Full bibliographic information for each citation is found at the end of the specification, immediately preceding the Claims. The disclosure of each of these references are incorporated by reference into the present disclosure).

The use of proteinaceous material of animal origin can result in the introduction of undesirable contaminants into the diphtheria toxin produced using such a medium.

The preparation of nutrient culture media from germinated seeds of Lupinus luteus (soybean extract) was used in a medium for the growth of C. diphtheriae by El Kholy et al 1967 (Ref 4). Although the bacteria grew well the production of diphtheria toxoid was minimal. El Kholy and Karamya (1979) (Ref. 5) concluded that sapponins in the soybean extract were an inhibitor of toxin production. Taha and Kholy (1985) (Ref. 6) autoclaved soybeans prior to successive extraction by boiling water to give an aqueous extract that yielded a toxin with an Lf value comparable to the control (meat broth), supposedly due to the destruction of the trypsin inhibitor by the steam autoclaving and reduction in the saponin content of the extracts by the successive boiling. Acidic extraction of soybean meal at pH 4.6 resulted in extracts with Lf values, which competed with Lf values of the control (meat broth), because both the saponins and the trypsin inhibitor are extracted in limited amounts at this pH.

International patent application WO 00/50449, published 31 Aug. 2000 of Wolfe et al and assigned to NYCOMED IMAGING AS describes media and a process for the production of diphtheria toxin. All media described in WO 00/50449 contain casamino acids which are obtained by the acid hydrolysis of the milk protein casein. Accordingly, all media described in WO 00/50449 contain proteinaceous material of animal origin.

International Patent Application WO 98/541296, published Dec. 3, 1998 of Oliveri et al and assigned to Chiron S.P.A. describes a medium for the production of diphtheria toxin that contains Soytone.

Analogs of diphtheria toxin have been described (see for example Ref 7) which are non-toxic and are often referred to as CRMs (cross-reacting materials). Examples of these are CRM-197, CRM-9, CRM-45, CRM-102, CRM-103 and CRM-107.

There remains a need for a bacterial growth medium substantially free or devoid of animal-components for the cultivation of C. diphtheriae and the production of diphtheria toxin and analogs thereof.

### SUMMARY OF THE INVENTION

The present invention is concerned with a growth medium and process for the production of diphtheria toxin and analogs thereof.

In a first aspect of the invention, there is provided a culture medium for producing diphtheria toxin or analog thereof wherein the medium is substantiality free of animal-derived products and comprises water; a carbohydrate source and a nitrogen source, a number of free amino acids in an initial concentration wherein the initial concentration of each free amino acid is not limiting for the level of production of the diphtheria toxin or the analog thereof. The culture medium may comprise all naturally occurring amino acids and the carbohydrate source may comprise maltose and the medium may be free of glucose. The nitrogen source may comprise yeast extract. The culture medium may be devoid of animal-derived products.

In a second aspect of the invention, there is provided a culture medium for Corynebacterium diphtheriae comprising a carbohydrate source and a nitrogen source and an additive system that comprises at least four free amino acids being each in an amount sufficient to promote a level of diphtheria toxin or analog thereof production by Corynebacterium diphtheriae wherein the medium is substantiality free of animal-derived products. The culture medium may comprise all naturally occurring amino acids and the carbohydrate source may be maltose. The nitrogen source may be yeast extract. Suitable amino acid concentrations are in the range from about to 0.5 grams to about 1 gram per litre of the medium. The culture medium may be devoid of animal-derived products.

In a third aspect of the invention, there is provided a method for the production of diphtheria toxin or analog thereof comprising the steps of culturing a strain of C. diphtheriae in any culture medium as provided herein. The C. diphtheriae strain may be grown until stationary phase and a production of at least 100 Lf/mL of diphtheria toxin or analog thereof may be obtained. The diphtheria toxin or analog thereof may be recovered, purified and detoxified to provide a diphtheria toxoid which may be formulated as a vaccine for immunizing a host against disease caused by infection by C. diphtheriae.

In a further aspect, the present invention extends to a method of immunizing a host against disease caused by infection by C. diphtheriae comprising administering the vaccine as provided herein to the host. Thus, the vaccine as provided herein can be used for immunizing a host against disease caused by infection by C. diphtheriae and the diphtheria toxoid as provided herein can be used in the preparation of a medicament for immunizing a host against disease caused by infection by C. diphtheriae.

In a further aspect the present invention provides a composition comprising a C. diphtheriae strain and a culture medium as provided herein

In a further aspect the present invention provides a method for producing diphtheria toxin or an analog thereof comprising growing a culture of Corynebacterium diphtheriae in a medium and providing at least one selected amino acid to the culture to prevent concentrations of the selected amino acids being limiting for toxin (or analog thereof) production wherein the medium is substantiality free of animal-derived products. The medium may further comprise a yeast extract at for example a concentration of about 3 g/L.

In a further aspect the present invention provides an improvement in a culturing method of Corynebacterium diphtheriae in a medium containing amino acids for producing a level of production of diphtheria toxin or an analog thereof and in which at least one selected amino acid is depleted during the culturing and limits the level of production of the diphtheria toxin or the analog thereof, the improvement comprising an exogenous addition of an additional amount of the at least one selected amino acid during said culturing and wherein the at least one selected amino acid is not limiting for the level of production of the diphtheria toxin or the analog thereof. The at least one selected amino acid may be selected from the group consisting of Glu, Asn, Ser, His, Gly, Thr, Met, Trp; and Isoleucine.

### BRIEF DESCRIPTION OF DRAWINGS

The present invention will be further understood from the following description with reference to the drawings, in which:

FIG. 1 is a graph showing the variable-interaction effects in the toxin yield by the yeast extract-amino acid interaction effect;

FIG. 2 is an SDS-PAGE analysis of diphtheria toxin and toxoid produced using the animal-component containing and animal-component free media;

FIG. 3 is a Western Blot analysis of diphtheria toxin and toxoid produced using the animal-component containing and animal-component free media;

FIG. 4 is a Isoelectric gel analysis of diphtheria toxin and toxoid produced using the animal-component containing and animal-component free media;

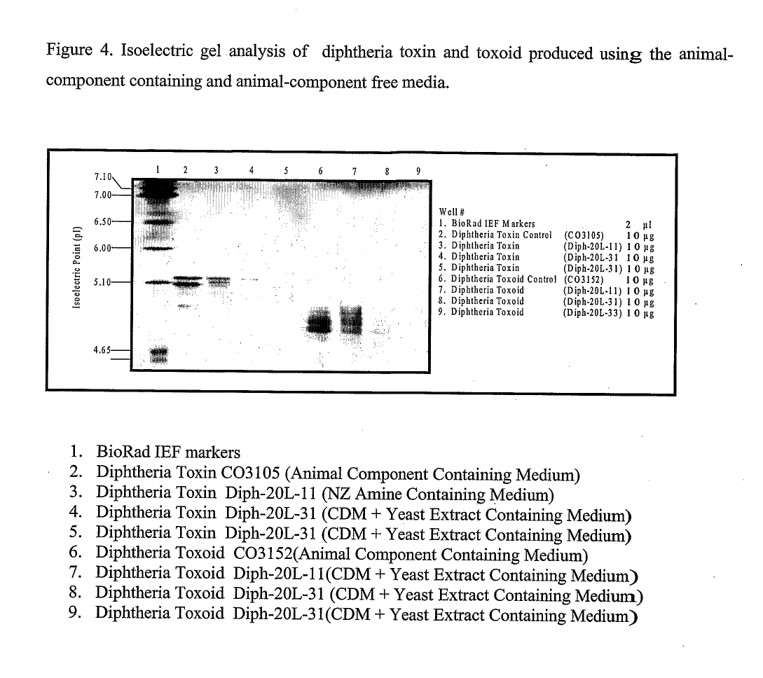

FIG. 5 shows the circular dichroism of diphtheria toxin produced using the animal-component containing and animal-component free media;

FIG. 6 shows the circular dichroism of diphtheria toxoid produced using the animal-component containing and animal-component free media; and

FIG. 7 shows the circular dichroism of diphtheria toxoid produced using the animal-component containing and animal-component free media at the 200 L scale.

### DETAILED DESCRIPTION OF THE INVENTION

### Formulation of Animal Component-Free Amino Acid Media

NZ Amine is a source of amino acids and peptides produced by the enzymatic digestion of casein. It is a good source of both amino nitrogen (free amino acids) and organic nitrogen (peptides). Another source of amino acids and peptides in C. diphtheriae media for the production of diphtheria toxoid is the animal-derived Toxiprotone-D. The compositions of these media are shown in Tables and below:

### Composition of the NZ Amine Containing Medium

TABLE 1
Composition of the NZ amine
Containing Medium

| Ingredient | Quantity per Liter | |
|---|---|---|
| NZ Amine | 30 | g |
| Acetic acid | 7.2 | mL |
| Maltose | 25 | g |
| Growth Factors | 8 | mL |
| 10% L-Cystine | 2 | Ml |
| 60% Sodium Lactate | 1.7 | Ml |
| PH | 7.5 | |

TABLE 2
Composition of the growth factor solution

| Ingredient | Quantity | |
|---|---|---|
| Magnesium sulphate | 225 | g |
| Beta Alanine | 2.30 | g |
| Pimelic acid | 0.15 | g |
| Zinc sulphate | 0.80 | g |
| Copper sulphate | 0.50 | g |
| Manganese chloride | 0.24 | g |
| Nicotinic acid | 4.6 | g |
| Hydrochloric acid, concentrated | 30 | mL |
| Water for Injection | 1000 | mL |

TABLE 3
Composition of the Toxiprotone Containing Medium

| Media component | Quantity (g/L) |
|---|---|
| Toxiprotone D | 52.5 g |
| SOLUTION DEXTROSE-AMINO ACIDS | |
| Beta cyclo dextrin | 1.7 |
| Glucose anhydrous | 125 |
| L-His | 5.55 |
| L-Asparagine | 27.7 |
| L-Glutamine | 8.3 |
| Glutamic acid | 2.8 |
| L-cysteine | 0.55 |
| Maltose-growth factor solution | 28.9 |

### Formulation of Animal Component-Free Amino Acid Media

In the preparation of the amino acid media, the approach was to select the higher concentration (mM) of each of the amino acids in both the Toxiprotone-D and the NZ-Amine animal constituent containing medium to produce a medium that could support growth and toxin production by C. diphtheriae.

C. diphtheriae was grown in NZ-Amine containing medium. Several amino acids were identified at different time intervals (24, 30 and 41 hours) to be consumed during fermentation (Table 4).

TABLE 4
Amino acid composition by HPLC in the Toxiprotone-D animal component (Toxiprotone-
D), and the NZ amine animal component media and the consumption of the
amino acids during fermentation using the NZ Amine medium.

| Samples | ASP | GLU | ASN | SER | GLN | HIS | GLY | THR | ALA | ARG | TYR |
|---|---|---|---|---|---|---|---|---|---|---|---|
| NZ Amine Diph-20L-11 T = 0 | 3.61 | 8.59 | 2.65 | 4.6 | 0 | 2.08 | 1.39 | 4.2 | 5.34 | 4.89 | 3.29 |
| Diph-20L-11 T = 24 | 0.21 | 1.45 | 0 | 0.3 | 0 | 1.85 | 3.34 | 2.91 | 11.7 | 3.85 | 2.72 |
| Diph-20L-11 T = 30 | 0.26 | 1.06 | 0 | 0.4 | 0 | 1.3 | 3.11 | 1.03 | 15.4 | 3.29 | 2.48 |
| Diph-20L-11 T = 41 | 0.17 | 0.95 | 0 | 0.2 | 0 | 0.53 | 0.7 | 0.21 | 9.67 | 2.14 | 1.32 |
| Toxiprotone D | 1.32 | 3.23 | 1.44 | 3.6 | 0.63 | 1.16 | 3.5 | 2.66 | 7.61 | 9.12 | 1.92 |

| Samples | VAL | MET | TRP | PHE | ILE | LEU | LYS | PRO |
|---|---|---|---|---|---|---|---|---|
| NZ Amine Diph-20L-11 T = 0 | 7.33 | 3.47 | 1.27 | 5.6 | 5.3 | 13.8 | 10 | 1.9 |
| Diph-20L-11 T = 24 hrs | 7.58 | 2.68 | 0.62 | 4.71 | 4.81 | 11 | 8.22 | 4.77 |
| Diph-20L-11 T = 30 hrs | 7.67 | 2.15 | 0.37 | 4.57 | 4.75 | 10.8 | 7.81 | 6.9 |
| Diph-20L-11 T = 41 hrs | 3.61 | 0.7 | 0 | 2.59 | 1.76 | 5.42 | 7.37 | 6.75 |
| Toxiprotone D Solution | 3.27 | 2.29 | 1.02 | 2.8 | 2.7 | 6.94 | 3.77 | 1.65 |

### Amino Acid Concentrations are in mM

By correlating the consumption of amino acids and toxin production, fermentation experiments were performed with the following medium containing the amino acids Asn, Glu, Ser, His, Gly, Thr, Met, Trp, Iso and Leu.

TABLE 5
Composition of the growth medium containing
the amino acids Asn, Glu, Ser,
His, Gly, Thr, Met, Trp, Iso and Leu.

| Ingredient | Quantity | |
|---|---|---|
| Acetic Acid | 7.2 | mL |
| Maltose | 25 | g |
| 60% Sodium Lactate | 1.7 | mL |
| Growth Factor Solution | 8 | mL |
| 10% L-Cystine | 2 | mL |
| (animal component-free) | | |
| L-Glutamic acid | 1 | g |
| L-Asparagine, Monohydrate | 0.5 | g |
| L-Serine | 0.5 | g |
| L-Histidine | 0.5 | g |
| L-Glycine | 0.5 | g |
| L-Threonine | 0.5 | g |
| L-Methionine | 0.5 | g |
| L-Tryptophan | 0.5 | g |
| L-Isoleucine | 1 | g |
| Water for Injection to 1000 mL | | |

However, using only these few amino acids did not result in cell growth or toxin production.

A medium (CDM) was devised that contained all of the naturally occurring amino acids. All the amino acids are from non-animal sources. The composition of this medium is shown below in Table 6 below.

TABLE 6
Composition of the animal component-free medium CDM.

| Ingredient | Quantity | |
|---|---|---|
| Acetic Acid | 7.2 | mL |
| Maltose | 25 | g |
| 60% Sodium Lactate | 1.7 | mL |
| Growth Factor Solution | 8 | mL |
| 10% L-Cystine (animal component-free) | 2 | mL |
| Yeast extract | 3 | g |
| L-Aspartic Acid | 0.5 | g |
| L-Glutamic acid | 1 | g |
| L-Asparagine, Monohydrate | 0.5 | g |
| L-Serine | 0.5 | g |
| L-Glutamine | 0.5 | g |
| L-Histidine | 0.5 | g |
| L-Glycine | 0.5 | g |
| L-Threonine | 0.5 | g |
| L-Arginine | 2 | g |
| L-Valine | 1 | g |
| L-Tryptophan | 0.5 | g |
| L-Phenylalanine | 1 | g |
| L-Isoleucine | 1 | g |
| L-Leucine | 2 | g |
| L-Lysine Hydrochloride | 2 | g |
| L-Proline | 2 | g |
| β-Alanine | 0.5 | g |
| L-Tyrosine | 0.5 | g |
| L-Methionine | 0.5 | g |
| Water for Injection to 1000 mL | | |

### Comparative Analysis of Fermentation Batches of C. Diphtheriae Strain Performed at the 20 L Scale Using NZ Amine or Toxiprotone Containing Medium or

### Fermentation Using the NZ Amine Containing Medium

In a first pre-Culture a lyophile seed was propagated from a lyophile seed to a Loefflers slant where the culture was grown for 22±2 hours at 36±2° C. In a second pre-Culture, after 22±2 hours of incubation, the cells from the slant were transferred to a primary flask of 100 mL of NZ Amine medium and incubated at 36±2° C. for 22 hours at 180 rpm. The flask also included 1 mL of a 1:10 diluted phosphate solution (32% (w/v)) and 0.5 mL of a 1:2 diluted calcium chloride solution (53% (w/v)). In a third pre-Culture about 5 mL of the primary culture was taken from the 100 mL primary shake flask and was inoculated into the 250 mL of NZ Amine medium and incubated for 22 hours at 36±2° C. and 180 rpm. The culture also included 2.5 mL of a 1:10 diluted phosphate solution (32% (w/v)) and 1.25 mL of a 1:2 diluted calcium chloride solution (53% (w/v)). In the fermentation, 15 mL of the third pre-culture was used to inoculate 15 L of NZ Amine medium in a fermentor. The culture also contained 100.7 mL of a 0.32% (w/v) phosphate solution and 125 mL of a 1:2 diluted calcium chloride solution (53% (w/v)) and 23.44 mL of ferrous sulfate heptahydrate solution (0.1% (w/v). The fermentation was carried out under controlled temperature of 36±2° C., in a Braun Fermentor with 1 Rushton turbine impeller, using agitation of 600 rpm, with aeration of 1.57 vvm through the headspace. After 25 hours of fermentation the agitation was increased to 800 rpm and the fermentor was pressurized to 0.4 bar. The fermentation was continued for another 16 hours.

### Fermentation Using the Toxiprotone Containing Medium

3.1.1 In a first pre-Culture a lyophile seed was propagated from a lyophile seed to a bacto-tryptose agar with 5% sheep blood agar plate and grown for 24±2 hours at 36±2° C. In a second pre-culture the cells from blood agar plate were transferred to a primary flask of 90 mL of medium and incubated for 48 hours stationary at room temperature and then for 24 hours at 180 rpm and 36±2° C. About 1.6 mL of the primary culture was taken from the 90 mL primary shake flask and inoculated into 800 mL of medium for 22 hours at 36±2° C. at 180 rpm. The 800 mL of culture was then used to inoculate 10 L of medium in the fermentor. The fermentation was carried out in a New Brunswick Scientific Fermentor with 2 Rushton turbine impellers, 1 sparger and 4 baffles. The culture was agitated at 220 rpm, with aeration of 0.2 vvm at 36±2° C. The pH was controlled between 7.5 to 7.6 using solution dextrose amino acid from 8 hours onwards until 32 hours of fermentation was completed. The Lf/mL generated was 80-90 Lf/mL.

### Fermentation Using the CDM Medium

### First Pre-Culture

A wet frozen seed (Glycerol stock) was propagated on a CDM+5 g/LYE agar medium and incubated at 36° C. for 24 hours.

### Second Pre-Culture

The culture on the plate was resuspended in 5 mL of CDM+3 g/LYE medium and 2.5 mL of it is used to inoculate the primary flask of 90 mL of the CDM+3 g/LYE medium. The flask was incubated under constant shaking at 200 rpm for 24 hours at 36° C. The primary flask also included 0.9 mL of a 1:10 diluted phosphate solution (32% (w/v)) and 0.45 mL of a 1:2 diluted calcium chloride solution (53% (w/v)).

### Fermentation

About 800 mL of the third pre-culture was used to inoculate. 10 L of CDM+3 g/L YE medium in the fermentor. 100 mL of a 1:10 diluted phosphate solution (32% (w/v)) and 50 mL of a 1:2 diluted calcium chloride solution (53% (w/v)) and 3.4 mL of ferrous sulfate heptahydrate solution (0.1% (w/v). were added to the fermentation. The fermentation was carried out under controlled temperature of 36° C. in New New Brunswick Scientific or B. Braun fermentor. The process parameters were: agitation of 250 rpm, aeration of 0.45 vvm. The pH was controlled between 6.5 to 7.6 using 5N sodium hydroxide and 2.5M phosphoric acid during the fermentation.

The amount of toxin quantified by flocculation method (Lf test) and ELISA (Table 7).

TABLE 7
Fermentations of C. diphtheriae in CDM.

| | 1° SF | | 2° SF | | | | | | |
|---|---|---|---|---|---|---|---|---|---|
| | Vol of | | Vol to | | Vol to | Fermentor | Lf/ | pH | |
| Batch No | Seed | OD | 2° SF | OD | Fermentor | Max OD | ml | Control | Remarks |
| Diph-20L-28 | 0.5 ml | 2.75 | 5 ml | 6.30 | 11 ml | 8.23 | — | 6.5-7.5 | CDM + 202 mL of 1:10 Phos + |
| | | | | | | | | | 55 mL of 1:2 CaCl₂ + 3.8 mL of |
| | | | | | | | | | 0.1% FeSO₄•7H₂O |
| | | | | | | | | | (11 L scale) |
| Diph-20L-29 | 0.5 ml | 2.75 | 5 ml | 6.30 | 11 ml | 3.12 | — | 7.0 | CDM + 202 mL of 1:10 Phos + |
| | | | | | | | | | 55 mL of 1:2 CaCl₂ + 3.8 mL of |
| | | | | | | | | | 0.1% FeSO₄•7H₂O |
| | | | | | | | | | (11 L scale) |
| Diph-20L-30 | 0.5 ml | 3.03 | 5 ml | 12.4 | 6 ml | 9.11 | — | — | CDM + 110 mL of 1:10 Phos + |
| | | | | | | | | | 30 mL of 1:2 CaCl₂ + 2 mL of |
| | | | | | | | | | 0.1% FeSO₄•7H₂O |
| | | | | | | | | | (6 L scale) |
| Diph-20L-31 | 0.5 ml | 3.03 | 5 ml | 12.4 | 6 ml | 10.63 | 120 | 6-8 | CDM + 110 mL of 1:10 Phos + |
| | | | | | | | | | 30 mL of 1:2 CaCl₂ + 2 mL of |
| | | | | | | | | | 0.1% FeSO₄•7H₂O |
| | | | | | | | | | (6 L scale) |

Fermentations were performed at the 240 L scale using the CDM medium with different combinations of the maltose, iron, and phosphate concentrations. The results are summarized in Table 8 below:

TABLE 8
240L fermentations using the animal component-free CDM

| | Concentrated | Calcium | 0.1% | | | |
|---|---|---|---|---|---|---|
| Maltose | phosphate | chloride | FeSO₄•7H₂O | Backpressure | Growth | |
| (g/L) | Vol. (L) | Vol.(L) | Vol.(L) | From (hrs) | OD₆₀₀ | Lf/mL |
| 25 | 0.44 | 0.6 | 0.2 | — | 15-20 | 60-100 |
| 25 | 0.44 | 0.6 | 0.083 | — | 15-20 | 60 |
| 25 | 2-2.5 | 1.0 | 0.25-0.45 | — | poor | None |
| 25 | 0.44 | 0.6 | 0.2 | 55 | 17 | 60 |
| 25 | 0.88 | 0.6 | 0.2 | 55 | 16 | 60 |
| 25 | 0.44 | 0.6 | 0.12 | 24 | 16 | 70 |
| 25 | 0.44 | 0.6 | 0.1 | 24 | 16 | 70 |
| 25 | 0.44 | 0.6 | 0.035 | 24 | 16 | 90 |
| 15 | 0.44 | 0.6 | 0.025-0.075 | 24 | 14 | 30-50 |
| 10 | 0.44 | 0.6 | 0.025 | 24 | 17 | 40 |

Although growth of OD₆₀₀ of 15-20 was achieved, the levels of toxin produced were 90-100 Lf/mL which is below the level obtained when a medium containing Proteinacious material of animal origin such as NZ amine or Phytone is used.

A time course study of amino acid consumption showed that the amino acids such as (Asp, Glu, Asn, Ser, Gln, Gly and Thr) were consumed within 12 hours of fermentation as shown in 20 L batches (Table 9) and are not available during the toxin expression phase

TABLE 9
Time course study of amino acid consumption in the CDM medium

| Sample | ASP | GLU | ASN | SER | GLN | HIS | GLY | THR | βALA | ALA |
|---|---|---|---|---|---|---|---|---|---|---|
| Diph-20L-31 | 4.53 | 6.42 | 2.02 | 4.02 | 2.95 | 2.75 | 5.63 | 3.66 | 12.65 | 0.16 |
| T = 0 | | | | | | | | | | |
| Diph-20L-31 | 0.17 | 7.43 | 0 | 0.13 | 0 | 2.60 | 5.05 | 2.80 | 13.65 | 1.48 |
| T = 31.5 | | | | | | | | | | |
| Diph-20L-31 | 0 | 0.72 | 0 | 0.17 | 0 | 1.75 | 0.30 | 0 | 12.81 | 1.47 |
| T = 49 | | | | | | | | | | |
| Diph-20L-31 | 0 | 0.97 | 0 | 0.19 | 0 | 1.48 | 0.30 | 0 | 12.54 | 0.92 |
| T = 56 | | | | | | | | | | |
| Diph-20L-31 | 0 | 0.69 | 0 | 0.21 | 0 | 1.16 | 0.35 | 0 | 12.02 | 0.62 |
| T = 71.5 | | | | | | | | | | |

| Sample | ARG | TYR | VAL | MET | TRP | PHE | ILE | LEU | LYS | PRO |
|---|---|---|---|---|---|---|---|---|---|---|
| Diph-20L-31 | 10.33 | 2.25 | 7.82 | 2.94 | 2.01 | 5.72 | 7.20 | 14.11 | 7.75 | 17.85 |
| T = 0 | | | | | | | | | | |
| Diph-20L-31 | 10.53 | 2.36 | 7.51 | 2.91 | 2.01 | 5.72 | 6.71 | 13.49 | 9.84 | 17.49 |
| T = 31.5 | | | | | | | | | | |
| Diph-20L-31 | 8.84 | 2.13 | 4.56 | 1.41 | 1.43 | 4.22 | 3.44 | 8.73 | 8.62 | 13.85 |
| T = 49 | | | | | | | | | | |
| Diph-20L-31 | 8.38 | 1.87 | 3.69 | 1.05 | 1.21 | 3.71 | 2.60 | 7.04 | 7.71 | 16.36 |
| T = 56 | | | | | | | | | | |
| Diph-20L-31 | 7.75 | 1.72 | 2.48 | 0.69 | 1.11 | 2.80 | 1.67 | 4.74 | 8.05 | 13.77 |
| T = 71.5 | | | | | | | | | | |

These results suggested that the medium should be enriched with either organic or inorganic nitrogen.

### Screening of Organic and Inorganic Nitrogen Supplements

A time course study of the amino acid consumption during the fermentation process showed that the key amino acids are consumed in the first 12-18 hours of growth and are not available in the later phase of fermentation when the toxin is produced. The medium should be supplemented with nitrogen to support the growth and in order to use amino acids in the medium as the precursors for the toxin synthesis. Yeast extract and ammonium sulfate were added to the CDM as described below:

The different media used for growth of C. diphtheriae and the production of diphtheria toxin were:
a) CDM+5 g/L of yeast extract;
b) CDM+5 g/L ammonium sulfate; and
c) A modified CDM containing half the concentration of amino acids in the medium+5 g/L yeast extract and 5 g/L ammonium sulfate.

The production of diphtheria toxin in these fermentations is shown in Table 10 below:

TABLE 10
Production of diphtheria toxin in CDM media
supplemented with organic and inorganic nitrogen.

| Medium | OD₆₀₀ T = 48 hr | OD₆₀₀ T = 72 hr | Lf/mL |
|---|---|---|---|
| (a) CDM | 5.61 | 4.18 | 40 |
| (b) CDM + 5 g/L YE | 4.48 | 5.17 | 40-60 |
| © CDM + 5 g/L (NH₄)₂ SO₄ | 7.44 | 8.10 | 40 |
| (d) CDM + 5 g/L YE + 5 g/L | 9.24 | 10.95 | 40 |
| (NH₄)₂ SO₄ | | | |

Optimization of different components in the medium using a statistical design to obtain higher toxin yield

A computer statistical design (FusionPro®) has been used to optimize the media composition. In the design, 3 components (yeast extract, amino acid mixture and iron) at 3 different concentrations are used as inputs in the statistical design. A fractional factorial design was chosen (see Table 11), below:

TABLE 11
The Experimental design varying the amount of yeast
extract, amino acid and iron concentrations to optimize
toxin production by C. diphtheriae

| | Yeast | Amino acid | Fe(mL/L) | DT |
|---|---|---|---|---|
| Run | extract | (concentration/ | What is this | production |
| No. | (g/L) | amount) | unit??? | (μg/mL) |
| 1 | 10 | 0.5 | 0 | 37.13 |
| 2 | 5 | 0.5 | 0.34 | 45.61 |
| 3 | 2.5 | 0.5 | 0.34 | 30.46 |
| 4 | 10 | 0* | 0.66 | 2.70 |
| 5 | 10 | 1 | 0.34 | 1.12 |
| 6 | 5 | 0.5 | 0.66 | 30.66 |
| 7 | 2.5 | 1 | 0.66 | 71.13 |
| 8 | 2.5 | 0* | 0 | 0.45 |
| 9 | 5 | 0* | 0.34 | 0.11 |
| 10 | 5 | 0.5 | 0.34 | 19.35 |
| 11 | 5 | 1 | 0 | 148.60 |

The phosphate and calcium chloride solutions are maintained as constant variables.

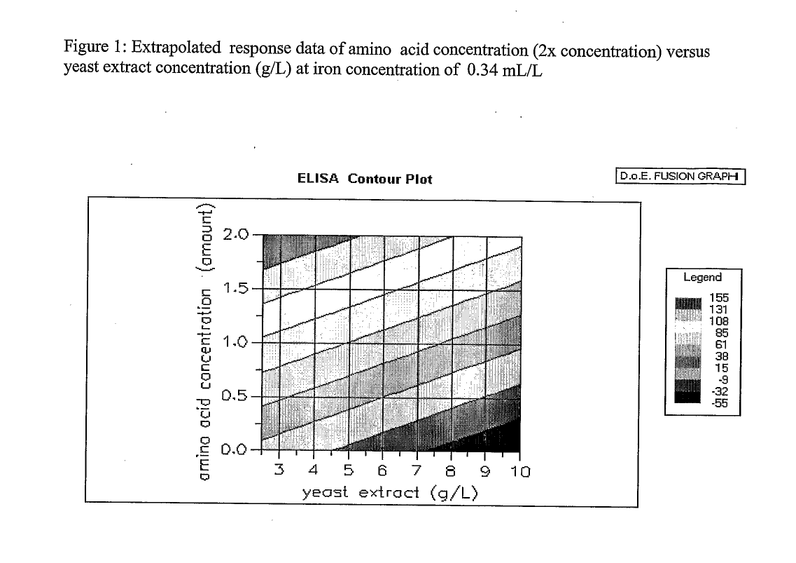

The experiment was performed under the different conditions and the amount of toxin produced was quantified by ELISA. Although the toxin concentration is around 150 Lf/mL, the toxin produced is purer than when the animal component is used in the fermentation process. Response graph of yeast extract and amino acid amount was extrapolated to double the concentration of the amino acid mixture with the iron concentration at 0.34 mL/L, as shown in FIG. 1. Under these conditions of yeast extract concentration (3 g/L) and amino acid concentration (2×), the amount of toxin is doubled according to the contour plot analysis. But in practice this cannot be readily implemented as it will increase the cost and also the osmolarity of the medium, leading to the death of the cells.

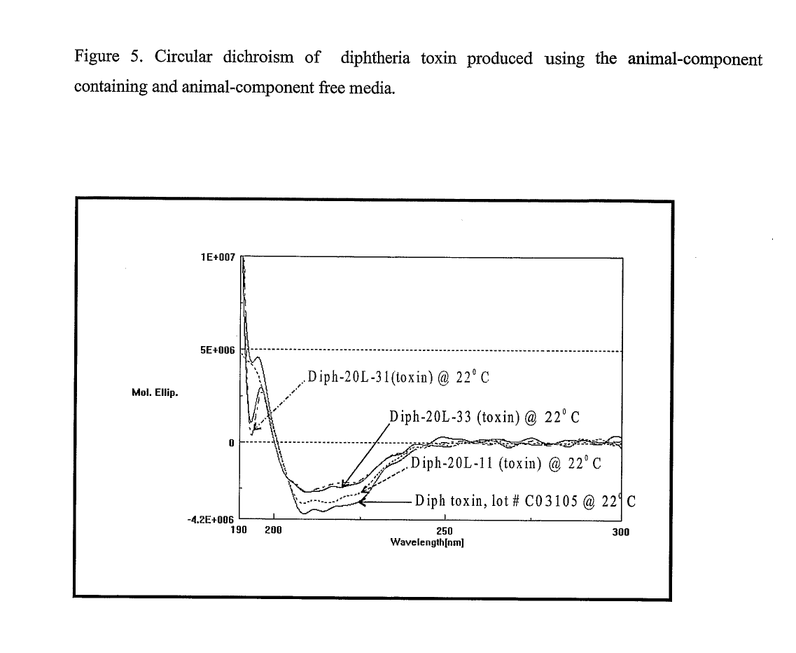

The statistical design has shown that there are important variable-interaction effects in the toxin yield. The most important effect (as shown in FIG. 1) is the yeast extract-amino acid interaction effect (A*B). Yeast extract and amino acid has a negative effect on toxin yield. If the yeast extract concentration is too high (i.e., 5 g/L), the conditions will support bacterial growth but not toxin production. Also if the amino acid concentration is increased to two fold, this may create an unfavourable environment for growth perhaps due to an imbalance in the osmotic pressure. Hence the yeast extract and amino acid concentrations have to be optimized for the production of high toxin concentrations. The general regression statistics in FIG. 5, show that the R square value is 0.92. This means that the observed toxin yield data is very close to the predicted toxin yield data generated by the FusionPro® design.

The optimum amount of toxin produced is at a yeast extract concentration of 3 g/L, an amino acid concentration of 1 fold and iron concentration at 0.34 mL/L.

Based on the earlier fermentation experiments and the profile of amino acid consumption during growth and toxin production phases, it was observed that the amino acids Asp, Glu, Asn, Ser, Gln, Gly and Thr are consumed faster and are not available during the toxin expression phase. These are the key amino acids that are being demanded in 2× concentration by the FusionPro extrapolated response plot instead of all the 19 amino acids, to achieve the higher yields of toxin. Hence a shake flask study of the effect of the 2× concentration of the key amino acids (Modified CDM1+3 g/L YE) on the toxin synthesis was conducted. Doubling the concentration of the above-mentioned key amino acids doubled the toxin levels (289 μg/mL) compared to 1× concentration (157 μg/mL) supporting the assumption that these amino acids are needed for the toxin synthesis that are being completely consumed during the growth. These conditions of 2× concentration of these amino acids were scaled-up in the 20 L fermentor and it was observed that the cell growth was poor. The optimized animal component-free medium was CDM+3 g/L of yeast extract as shown below in Table 12.

TABLE 12
Composition of CDM + 3 g/L of yeast extract Medium

| Ingredient | Quantity | |
|---|---|---|
| Acetic Acid | 7.2 | mL |
| Maltose | 25 | g |
| 60% Sodium Lactate | 1.7 | mL |
| Growth Factor Solution | 8 | mL |
| 10% L-Cystine (animal component-free) | 2 | mL |
| Bacto-Yeast extract | 3 | g |
| L-Aspartic Acid | 0.5 | g |
| L-Glutamic acid | 1 | g |
| L-Asparagine, Monohydrate | 0.5 | g |
| L-Serine | 0.5 | g |
| L-Glutamine | 0.5 | g |
| L-Histidine | 0.5 | g |
| L-Glycine | 0.5 | g |
| L-Threonine | 0.5 | g |
| L-Arginine | 2 | g |
| L-Valine | 1 | g |
| L-Tryptophan | 0.5 | g |
| L-Phenylalanine | 1 | g |
| L-Isoleucine | 1 | g |
| L-Leucine | 2 | g |
| L-Lysine Hydrochloride | 2 | g |
| L-Proline | 2 | g |
| β-Alanine | 0.5 | g |
| L-Tyrosine | 0.5 | g |
| L-Methionine | 0.5 | g |
| Water for Injection | to 100 | mL |

### Purification and Detoxification of Diphtheria Toxoid

Ten liters of culture from a fermentation was centrifuged at 12,500×g for 20 minutes at 4° C. and the supernatant was collected. The supernatant was then filtered through a 0.22 μm membrane filter to remove residual bacteria. Ammonium sulfate 27% (w/v) of was added to the filtered supernatant under constant stirring at 4° C. and then centrifuged at 12,500 g for 20 minutes at 4° C. The supernatant was collected for further processing. Ammonium sulfate 13% (w/v) was added to this supernatant under constant stirring. The mixture was further stirred overnight at 4° C. and then centrifuged at 12,500 g for 20 minutes at 4° C. The resultant pellet was dissolved in about 1000 mL of 0.9% (w/v) saline.

The above toxin solution was diafiltered against 0.9% (w/v) saline using an ultrafiltration unit with a 10 kDa cassette to eliminate the ammonium sulfate. The retentate was filtered through 0.22 μm membrane filter and stored at 4-8° C. The retentate was diluted to 500 Lf/ml with 0.9% (w/v) saline prior to detoxification. The diphtheria toxin was at least 75% pure. Formalin 0.5% (v/v) and 0.5% (w/v) sodium bicarbonate were added to the diluted toxin solution under constant stirring at room temperature for 20 min. After 20 min 0.913% (w/v) L-lysine solution in 0.9% (w/v) saline was added and the mixture filtered through 0.22 μm membrane filter and incubated at 37° C. for 6 weeks under constant shaking for detoxification. The toxoid was stored at 4-8° C.

### Characterization of the Diphtheria Toxin and Toxoid Produced Using the Animal-Component Containing and Animal-Component Free Media

The diphtheria toxin and toxoid produced using the animal-component containing and animal-component free media were analyzed on SDS-PAGE, Western Blot, a determination of the CD spectra, N-terminal sequencing. The results indicate that both the toxin and toxoid obtained using the using the animal-component containing and animal-component free media were essentially indistinguishable.

Total protein concentration was preformed using bicinchoninic acid (BCA) in a microplate BCA assay and by comparison with a reference standard protein of known concentration.

### SDS PAGE

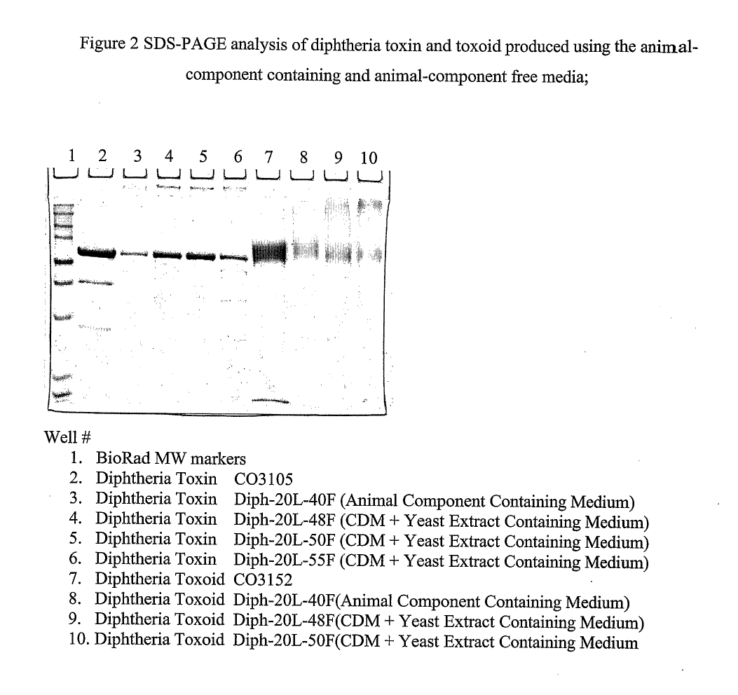

SDS-PAGE was preformed to determine relative molecular weight (Mᵣ) of diphtheria toxin and toxoid, to assess the purity of toxin and toxoid; and to evaluate the distribution patterns of the protein bands. Proteins are analyzed by SDS-PAGE on a 12.5% polyacrylamide gel under reducing conditions. The gel is stained with Coomassie Blue, followed by densitometry analysis. Referring to FIG. 2, there is shown an SDS-PAGE performed to determine relative molecular weight (Mᵣ) of diphtheria toxin and toxoid, to assess the purity of toxin and toxoid and to evaluate the distribution patterns of the protein bands. Proteins were analyzed by SDS-PAGE on a 12.5% polyacrylamide gel under reducing conditions. The gel was stained with Coomassie Blue, followed by densitometry analysis. The lanes are 1. MW markers (kDa), 250, 150, 100, 75, 50, 37, 25, 15, 10 kDa; 2. Diphtheria Toxin, CO3105 (Animal Component Containing Medium); 3. Diphtheria Toxin Diph-20L-40F (Animal Component Containing Medium); 3. Diphtheria Toxin Diph-20L-48F (CDM+Yeast Extract Containing Medium); 4. Diphtheria Toxin Diph-20L-50F (CDM+Yeast Extract Containing Medium); 5. Diphtheria Toxin Diph-20L-55F (CDM+Yeast Extract Containing Medium); 6. Diphtheria Toxoid CO3152; 7. Diphtheria Toxoid Diph-20L-40F (Animal Component Containing Medium); 8. Diphtheria Toxoid Diph-20L-48F (CDM+Yeast Extract Containing Medium); 9. Diphtheria Toxoid Diph-20L-50F (CDM+Yeast Extract Containing Medium)

### Western Blot Analysis

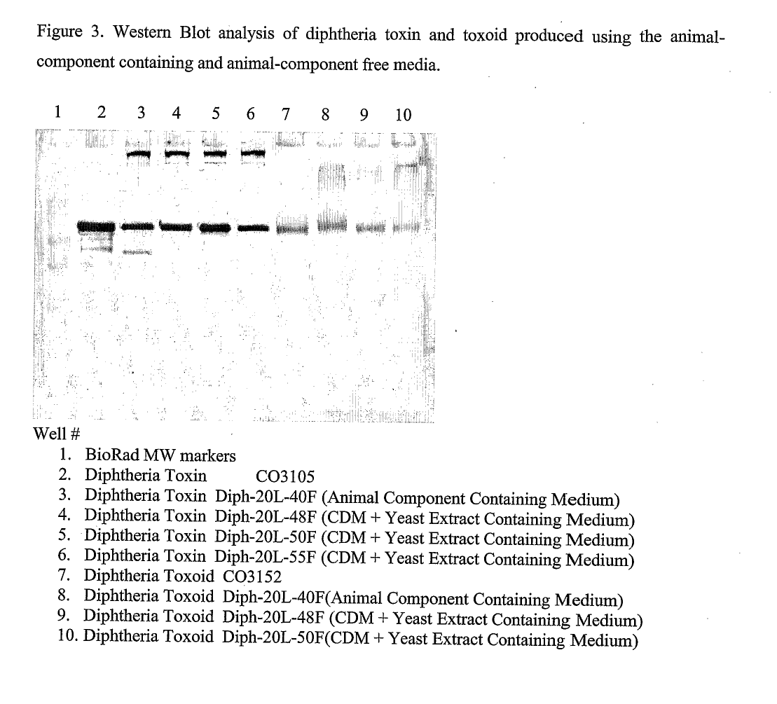

Referring to FIG. 3, there is shown a Western blot analysis using a diphtheria toxin specific antibody. Samples were resolved on 12.5% SDS-PAGE gels, transferred to a PVDF membrane, and blotted with a DT-specific antibody. The lanes are 1. Relative molecular weight markers (kDa), 250, 150, 100, 75, 50, 37, 25, 15, 10 kDa; BioRad MW markers; 2. Diphtheria Toxin CO3105; 3. Diphtheria Toxin Diph-20L-40F (Animal Component Containing Medium); 4. Diphtheria Toxin Diph-20L-48F (CDM+Yeast Extract Containing Medium); 5. Diphtheria Toxin Diph-20L-50F (CDM+Yeast Extract Containing Medium); 6. Diphtheria Toxin Diph-20L-55F (CDM+Yeast Extract Containing Medium); 7. Diphtheria Toxoid CO3152; 8. Diphtheria Toxoid Diph-20L-40F (Animal Component Containing Medium); 9. Diphtheria Toxoid Diph-20L-48F (CDM+Yeast Extract Containing Medium); 10. Diphtheria Toxoid Diph-20L-50F (CDM+Yeast Extract Containing Medium)

### N-Terminal Sequencing

N-terminal sequence analysis was used to monitor any protein modifications resulting in changes of the N-terminus. The proteins were resolved on a 12.5% SDS-Page gel and transferred to a solid support such as PVDF. The N-terminal amino acids are released and derivatized by the traditional Edman degradation process prior to identification by reversed-phase high performance liquid chromatography (RP-HPLC). The expected N-terminal sequences were observed for the manufacturing controls for diphtheria toxin and toxoid, as well as the ‘animal-free’ toxin/toxoid.

TABLE 13
N-terminal sequence of diphtheria toxin

| Lot # | Medium | Sequence | Matches |
|---|---|---|---|
| CO3105 | NZ Amine | GADDVVDSSKSF | Diphtheria Toxin protein |
| | | | GADDVVDSSKSF |
| Diph-20L-11 | NZ Amine | GADDVVDSSKSF | Diphtheria Toxin protein |
| | | | GADDVVDSSKSF |
| Diph-20L-31 | CDM + Yeast | GADDVVDSSKSF | Diphtheria Toxin protein |
| | Extract (15 g/L) | | GADDVVDSSKSF |
| Diph-20L-33 | CDM + Yeast | GADDVVDSSKSF | Diphtheria Toxin protein |
| | Extract (15 g/L) | | GADDVVDSSKSF |
| Diph-20L-64 | CDM + Yeast | GADDVVDSSKSF | Diphtheria Toxin protein |
| | Extract (15 g/L) | | GADDVVDSSKSF |

TABLE 14
N-terminal sequence of diphtheria toxoid

| Lot # | Medium | Sequence | Matches |
|---|---|---|---|
| CO3152 | NZ Amine | GADDVVDSSKSF | Diphtheria Toxin protein precursor |
| | | | GADDVVDSSKSF |
| Diph-20L-11 | NZ Amine | GAD-VVDSSKSF | Diphtheria Toxin protein precursor |
| | | | GADDVVDSSKSF |
| Diph-20L-64 | CDM + | GADDVVD | Diphtheria Toxin protein precursor |
| | Yeast Extract | | GADDVVDSSKSF |
| | (15 g/L) | | |

-missed sequence cycles due to instrument problems

### Isoelectric Focusing (IEF)

The isoelectric point of the diphtheria toxin was estimated with the use of a reference proteins. Referring to FIG. 3, there is shown an Isoelectric focusing gel. The lanes are: 1. IEF stds—pI=7.80, 7.50, 7.10, 7.00, 6.50, 6.00, 5.10, 4.65; 2. Diphtheria Toxin CO3105 (Animal Component Containing Medium); 3. Diphtheria Toxin Diph-20L-11 (NZ Amine Containing Medium); 4. Diphtheria Toxin Diph-20L-31 (CDM+Yeast Extract Containing Medium); 5. Diphtheria Toxin Diph-20L-31 (CDM+Yeast Extract Containing Medium); 6. Diphtheria Toxoid CO3152 (Animal Component Containing Medium); 7. Diphtheria Toxoid Diph-20L-11 (CDM+Yeast Extract Containing Medium); 8. Diphtheria Toxoid Diph-20L-31 (CDM+Yeast Extract Containing Medium); 9. Diphtheria Toxoid Diph-20L-31 (CDM+Yeast Extract Containing Medium)

### CD Spectroscopy

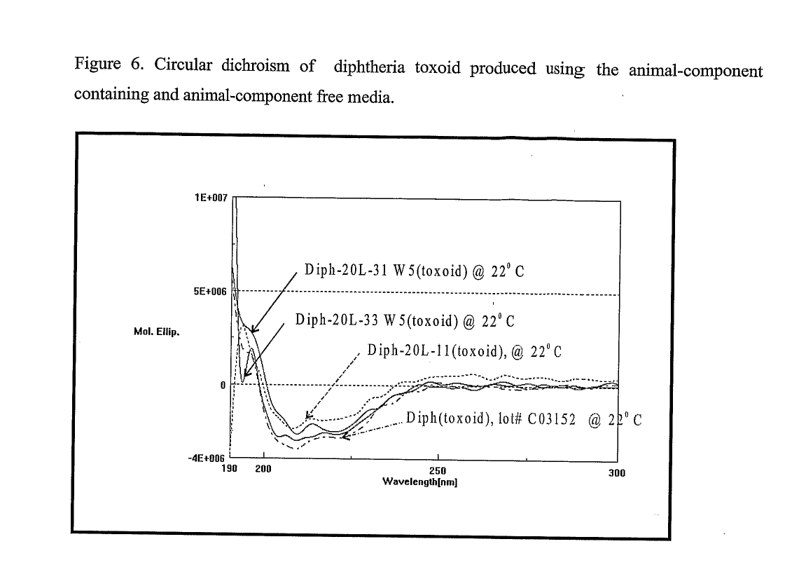

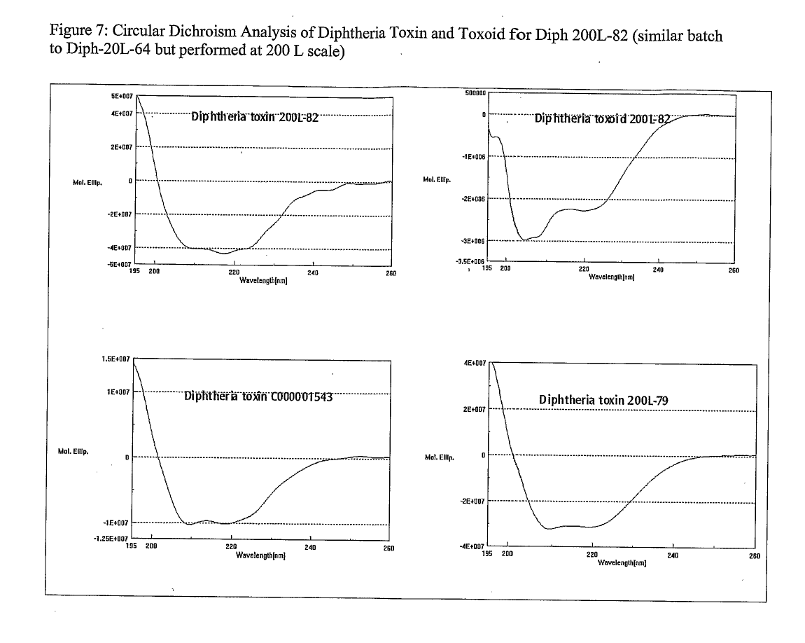

Circular Dichroism (CD) analysis was used to determine inconsistencies in the conformation or secondary structures of various lots. The absorbance spectrum for circularly polarized light of the sample is analyzed by a software program to yield a relative percentage composition of alpha-helix, beta-sheet, reverse-turn and random coil structure. Diphtheria toxin and toxoid were analyzed at 22° C. using a Jasco CD Spectropolarimeter. (see FIGS. 5-7).

N-terminal sequencing. The proteins were resolved on a 12.5% SDS-PAGE gel and transferred to a solid support such as PVDF. The N-terminal amino acids are released and derivatized by the traditional Edman degradation process prior to identification by reversed-phase high performance liquid chromatography (RP-HPLC).

### REFERENCES

1. Sundaran, B., Udaya, Y., Rao, B. and Boopathy, R. (2001) Process optimization for enhanced production of diphtheria toxin by submerged cultivation. Journal of Bioscience and Bioengineering 91, No. 2, 123-128.
2. Stainer, D. W. and Scholte, M. J. (1973). The production of high potency diphtheria toxin in submerged culture in relatively simple equipment using a semisynthetic medium. Biotechnology and Bioengineering Symposium. No. 4, 283-293.
3. Zaki, A. M. (1971) Production of diphtheria toxin in submerged culture. The Journal of the Egyptian Public Health Association 46, No. 2, 80-85.
4. El Kohly S., Shaheen Y., and Abdel Fattah, F. (1967). A new modificcation of lupinus culture medium. The Journal of the Egyptian Public Health Association 42, 1-7.
5. El Kohly S., and Karawya M. S (1979). Preliminary phytochemical and microbiological screening of Lupinus termis Forsk seeds. Bulletin of Faculty of Pharmacy, Cairo Universtiy XVIII No. 2:9-15.
6. Taha, F. S. and Kholy, S. E. (1985). Soybean extracts as culture media for the growth and toxin production of coryneabacterium diphtheriae. The Journal of the Egyptian Public Health Association 60: 113-126.
7. Nicholls and Youle in Genetically Engineered Toxins Ed: Frankel, Marcel Dekker Inc., 1992.

1. A culture medium for culturing a strain of Corynebacterium diphtheriae to produce a level of diphtheria toxin or an analog thereof wherein the medium is substantiality free of animal-derived products and comprises: water; a carbohydrate source; a nitrogen source; and a number of free amino acids in an initial concentration wherein the initial concentration of each free amino acid is not limiting for the level of production of the diphtheria toxin or the analog thereof.

2. The culture medium of claim 1 comprising all naturally occurring amino acids.

3. The culture medium of claim 1 wherein the carbohydrate source comprises maltose.

4. The culture medium of claim 1 substantially free of glucose.

5. The medium of claim 1 wherein the nitrogen source comprises yeast extract.

6. The culture medium of claim 1 wherein the medium is devoid of animal-derived products.

7. A medium for Corynebacterium diphtheriae comprising: a carbohydrate source and a nitrogen source and an additive system that comprises at least four free amino acids being each in an amount sufficient to promote a level of diphtheria toxin or an analog thereof production by Corynebacterium diphtheriae wherein the medium is substantiality free of animal-derived products.

8. The culture medium of claim 7 comprising all naturally occurring amino acids.

9. The medium of claim 7, wherein the carbohydrate source is maltose.

10. The medium of claim 7, wherein the nitrogen source is yeast extract.

11. The medium of claim 7 wherein amino acid concentrations are in the range from about to 0.5 grams to about 1 gram per litre of the medium.

12. The medium of claim 7 wherein the medium is devoid of animal-derived products.

13. A method for the production of diphtheria toxin or an analog thereof comprising the steps of: culturing a strain of C. diphtheriae in a culture medium under conditions that allow production of diphtheria toxin, wherein the culture medium is substantially free of animal-derived products and comprises: water; a carbohydrate source; a nitrogen source; and a number of free amino acids in an initial concentration wherein the initial concentration of each free amino acid is not limiting for the level of production of the diphtheria toxin or the analog thereof.

14. The method of claim 13, wherein the culture medium comprises all naturally occurring amino acids.

15. The method of claim 13, wherein the carbohydrate source comprises maltose.

16. The method of claim 13, wherein the culture medium is substantially free of glucose.

17. The method of claim 13, wherein the nitrogen source comprises yeast extract.

18. The method of claim 13 wherein the C. diphtheriae strain is grown until stationary phase.

19. The method of claim 13 wherein a production of at least 100 Lf/mL of diphtheria toxin or analog thereof is obtained.

20. The method of claim 13 further comprising a step of recovering the diphtheria toxin or analog thereof to provide a recovered diphtheria toxin or analog thereof.

21. The method of claim 17 further comprising a step of purifying the recovered diphtheria toxin or analog thereof to provide a purified diphtheria toxin or analog thereof.

22. The method of claim 17 or 18 further comprising a step of detoxifying the recovered or purified diphtheria toxin or analog thereof to provide a diphtheria toxoid or analog thereof.

23. The method of claim 19 further comprising formulating the diphtheria toxoid or analog thereof as a vaccine for immunizing a host against disease caused by infection by C. diphtheriae.

24. The method of claim 13 wherein the medium is devoid of animal-derived products.

25. A method of immunizing a host against disease caused by infection by C. diphtheriae comprising administering the vaccine of claim 23 to the host.

26. The use of the vaccine of claim 23 for immunizing a host against disease caused by infection by C. diphtheriae.

27. The use of the diphtheria toxoid or analog thereof of claim 22 in the preparation of a medicament for immunizing a host against disease caused by infection by C. diphiheriae.

28. A composition comprising a C. diphtheriae strain and a culture medium for producing diphtheria toxin or analog thereof wherein the medium is substantially free of animal derived products and comprises wherein the culture medium is substantially free of animal-derived products and comprises: water; a carbohydrate source; a nitrogen source; and a number of free amino acids in an initial concentration wherein the initial concentration of each free amino acid is not limiting for the level of production of the diphtheria toxin or the analog thereof.

29. The composition of method of claim 28, wherein the culture medium comprises all naturally occurring amino acids.

30. The composition of method of claim 28, wherein the carbohydrate source comprises maltose.

31. The composition of claim 28, wherein the culture medium is substantially free of glucose.

32. The composition of method of claim 28, wherein the nitrogen source comprises yeast extract.

33. The culture medium of claim 28 wherein the medium is devoid of animal-derived products.

34. A method for producing diphtheria toxin or an analog thereof comprising growing a culture of Corynebacterium diphtheriae in a medium and providing at least one selected amino acid to the culture to prevent concentrations of the selected amino acids being limiting for toxin production wherein the medium is substantiality free of animal-derived products.

35. The method of claim 34 wherein the medium further comprises a yeast extract.

36. The method of claim 35 wherein the yeast extract is present at a concentration of about 3 g/L concentration of amino acids.

37. In a culturing method of Corynebacterium diphtheriae in a medium containing amino acids for producing a level of production of diphtheria toxin or an analog thereof and in which at least one selected amino acid is depleted during the culturing and limits the level of production of the diphtheria toxin or the analog thereof, the improvement comprising an exogenous addition of an additional amount of the at least one selected amino acid during said culturing and wherein the at least one selected amino acid is not limiting for the level of production of the diphtheria toxin or the analog thereof.

38. The method of claim 37 wherein the at least one selected amino acid is selected from the group consisting of Glu, Asn, Ser. His, Gly, Thr, Met, Trp, and Isoleucine.

39. The method of claim 37, wherein the medium comprises a yeast extract.

40. The method of claim 39 wherein the yeast extract is present at a concentration of about 3 g/L.

41. The culture medium of claim 2 wherein the medium is devoid of animal-derived products.

42. The culture medium of claim 3 wherein the medium is devoid of animal-derived products.

43. The culture medium of claim 4 wherein the medium is devoid of animal-derived products.

44. The culture medium of claim 5 wherein the medium is devoid of animal-derived products.

45. The medium of claim 8 wherein amino acid concentrations are in the range from about to 0.5 grams to about 1 gram per litre of the medium.

46. The medium of claim 9 wherein amino acid concentrations are in the range from about to 0.5 grams to about 1 gram per litre of the medium.

47. The medium of claim 10 wherein amino acid concentrations are in the range from about to 0.5 grams to about 1 gram per litre of the medium.

48. The medium of claim 8 wherein the medium is devoid of animal-derived products.

49. The medium of claim 9 wherein the medium is devoid of animal-derived products.

50. The medium of claim 10 wherein the medium is devoid of animal-derived products.

51. The medium of claim 11 wherein the medium is devoid of animal-derived products.

52. The method of claim 14 wherein the medium is devoid of animal-derived products.

53. The method of claim 15 wherein the medium is devoid of animal-derived products.

54. The method of claim 16 wherein the medium is devoid of animal-derived products.

55. The method of claim 17 wherein the medium is devoid of animal-derived products.

56. The method of claim 18 wherein the medium is devoid of animal-derived products.

57. The method of claim 19 wherein the medium is devoid of animal-derived products.

58. The method of claim 20 wherein the medium is devoid of animal-derived products.

59. The method of claim 21 wherein the medium is devoid of animal-derived products.

60. The method of claim 22 wherein the medium is devoid of animal-derived products.

61. The method of claim 23 wherein the medium is devoid of animal-derived products.

62. The culture medium of claim 29 wherein the medium is devoid of animal-derived products.

63. The culture medium of claim 30 wherein the medium is devoid of animal-derived products.

64. The culture medium of claim 31 wherein the medium is devoid of animal-derived products.

65. The culture medium of claim 32 wherein the medium is devoid of animal-derived products.
